(19) United States
(12) Patent Application Publication
(10) Pub. No.: US 2024/0019441 A1
(43) Pub. Date: Jan. 18, 2024

(54) SETDB1-MICROTUBULE INTERACTION AND USE THEREOF

(71) Applicant: ARIEL SCIENTIFIC INNOVATIONS LTD., Ariel (IL)

(72) Inventor: Gabi GERLITZ, Tel-Aviv (IL)

(21) Appl. No.: 18/227,122

(22) Filed: Jul. 27, 2023

Related U.S. Application Data
(63) Continuation of application No. PCT/IL2022/050120, filed on Jan. 27, 2022.
(60) Provisional application No. 63/142,566, filed on Jan. 28, 2021.

(51) Int. Cl. G01N 33/574; C12Q 1/6886; A61K 31/337; A61K 31/475; A61P 35/00

(52) U.S. Cl. CPC G01N 33/57496; C12Q 1/6886; A61K 31/337; A61K 31/475; A61P 35/00; C12Q 2600/106; C12Q 2600/158; G01N 2333/91011

(57) ABSTRACT

Methods of determining suitability of a subject to be treated with a microtubule targeting agent (MTA) by measuring SETDB1 expression levels are provided. Methods of treating cancer by administering an MTA are also provided. Kits comprising agents for specific detection of SETDB1 are also provided.

### CROSS-REFERENCE TO RELATED APPLICATIONS

This application a bypass Continuation of PCT Patent Application No. PCT/IL2022/050120 having International filing date of Jan. 27, 2022, which claims the benefit of priority of U.S. Provisional Patent Application No. 63/142,566 filed Jan. 28, 2021, the contents of which are incorporated herein by reference in their entirety.

### FIELD OF INVENTION

The present invention is in the field of cancer therapeutics and diagnostics.

### BACKGROUND OF THE INVENTION

SETDB1 (also known as ESET and KMT1E) is a lysine methyltransferase (KMT) that belongs to the SUV39 family of KMTs that mainly methylate lysine 9 in histone H3 (H3K9). The SUV39 family members are characterized by cysteine-rich pre- and post-SET domains flanking a central SET domain that is responsible for the catalytic activity. SETDB1 also contains a methyl-CpG-binding domain (MBD) and a triple Tudor domain responsible for binding of H3K9me/K14ac to promote histone deacetylation by HDACs. SETDB1 can mono-, di- and tri-methylate H3K9. H3K9me2/3 are usually associated with gene repression and heterochromatin formation, and indeed SETDB1 is involved in silencing the X chromosome, repetitive elements and several specific genes. SETDB1 is important for various developmental processes including embryogenesis, neurogenesis, immune cell development, germ line development and chondrocyte differentiation. SETDB1 was also shown to methylate non-histone proteins such as ING2, p53, UBF and Akt.

SETDB1 methylation of H3K9 as well as most of its non-histone targets that are nuclear proteins, is in correlation with its nuclear localization. However, a cytoplasmic pool of SETDB1 has been found in several types of cells including HeLa cells, HEK293 cells, mouse embryonic fibroblasts, differentiated myoblasts and human melanoma biopsies. Cytoplasmic localization of SETDB1 is thought to facilitate methylation of newly synthesized histones before their incorporation into nucleosomes or to restrict the enzyme of methylating nucleosomal H3K9.

In recent years SETDB1 has been defined as an oncogene. Its genomic location is commonly amplified in melanoma and its expression levels are increased in various types of cancer including melanoma, colorectal cancer, liver cancer and lung cancer. SETDB1 oncogenic behavior supports cancer cell proliferation, migration and invasion. More recently SETDB1 was also linked to adaptive resistance of tumor cells to various drugs and repression of the innate immune response.

Currently SETDB1 is thought to promote cancer mainly by its nuclear activity of methylating H3K9 or transcription factors such as p53. However, since a substantial amount of SETDB1 can be found in the cytoplasm understanding of the role of this pool of SETDB1 is greatly needed. In particular, targeting therapies based on SETDB1 expression to produce patient-specific treatment regimens is of great importance.

### SUMMARY OF THE INVENTION

The present invention provides methods of determining suitability of a subject to be treated with a microtubule targeting agent (MTA) by measuring SETDB1 expression levels. Kits comprising agents for specific detection of SETDB1 are also provided.

According to a first aspect, there is provided a method of determining suitability of a subject in need thereof to be treated with a microtubule targeting agent (MTA), the method comprising receiving a sample from the subject, measuring SETDB1 expression in the sample and determining suitability based on the SETDB1 expression, thereby determining suitability of a subject to be treated with an MTA.

According to some embodiments, the MTA is a microtubule (MT) stabilizing agent and wherein expression of SETDB1 above a predetermined threshold indicates the subject is unsuitable for treatment with the MTA.

According to some embodiments, the MTA is an MT stabilizing agent and wherein expression of SETDB1 at or below a predetermined threshold indicates the subject is suitable for treatment with the MTA.

According to some embodiments, the MT stabilizing agent is selected from plant-based MT stabilizing agent, a bacterial MT stabilizing agent and a small molecule stabilizing agent.

According to some embodiments, the MT stabilizing agent is selected from a taxane, a taccalonalide, an epothilone, ceratamine A, ceratamine B, FR182877, dictyostatin, discodermolide, KS-1-199-32, eleutherobin, sarcodictyin, A, sarcodictyin B, zampanolide, dactylolide, laulimalide, isolaulimalide, peloruside A, persin, GS-164, Synstab, 4′-methoxy-2-styrylchromone, (Z)-1-(2-bromo-3, 4, 5-trimethoxyphenyl)-3-(3-hydroxy-4-methoxyphenylamino)prop-2-en-1-one, an a-cyano bis(indolyl)chalcone, a dienone and a derivative thereof.

According to some embodiments, the taxane is selected from paclitaxel, paclitaxel C, docetaxel, abraxane, cabazitaxel, larotaxel tesetaxel, TPI-287, 10-deacetylbaccatin III, baccatin III, and 7-epipaclitaxe and derivatives thereof.

According to some embodiments, the taccalonalide is selected from taccalonalide A, B, E, H2, I, J, N, R, T, Z, AA, AB, AC, AD, AF, AJ, AK, AL, AM, AN and AO.

According to some embodiments, the epothilone is selected from epothilone A, epothilone B, epothilone C, epothilone D, epothilone E, ixabepilone, sagopilone, patupilone, BMS-310705, KOS-1584 and BMS-753493.

According to some embodiments, the MT stabilizing agent is an HDAC6 inhibitor.

According to some embodiments, the MTA is an MT destabilizing agent and wherein expression of SETDB1 above a predetermined threshold indicates the subject is suitable for treatment with the MTA.

According to some embodiments, the MTA is an MT destabilizing agent and wherein expression of SETDB1 at or below a predetermined threshold indicates the subject is unsuitable for treatment with the MTA.

According to some embodiments, the MT destabilizing agent is selected from plant-based MT destabilizing agent, a bacterial MT destabilizing agent and a small molecule destabilizing agent.

According to some embodiments, the MT destabilizing agent is selected from a vinca alkaloid, maytansine, disorazole Z, a lactone, circumin, colchicine, combretastatin, TH588, chalcone, podophyllotoxin, indibulin, is 2-methoxyestradiol, pironetin, noscapinoid, noscapine, 9-bromonoscapine, quercetin and derivatives thereof.

According to some embodiments, the vinca alkaloid is selected from vinblastine, vincristine, colcemid, nocodazole, vindesine, vinorelbine, vincaminol, vineridine, vinburnine, vinpocetine, vinflunine, minovincine, methoxyminovincine, minovincinine, vincdifformine, desoxyvincaminol, crypthophycin1, romidepsin (FK-228), halichondrin B, erilubin, soblidotin, dolastin 15, dolastin 10, and vincamajine.

According to some embodiments, the lactone is selected from plocabulin (PM060184), emtansine, rhizoxin, and spongistatin.

According to some embodiments, the derivative of combretastatin is selected from combretastatin A-1, combretastatin A-4, fosbretabulin, Oxi4503, combretastatin 4-O-phosphate (CA-4P), ombrabulin.

According to some embodiments, the MT destabilizing agent is HDAC6 or a functional fragment thereof.

According to some embodiments, the predetermined threshold is an expression level in a healthy control.

According to some embodiments, the subject suffers from a disease, disorder or condition treatable by an MTA.

According to some embodiments, the disease is a proliferative disease.

According to some embodiments, the proliferative disease is cancer.

According to some embodiments, the cancer is treatable with MTAs or is known to over express SETDB1.

According to some embodiments, the cancer is selected from brain, skin, breast, lung, renal, liver, pancreatic, head and neck, hematopoietic, endometrial, bladder, sarcoma, glioma, colorectal, gastric, prostate, ovarian, testicular, and cervical cancer.

According to some embodiments, the sample is a tumor sample, comprises tumor cells or comprises cell free DNA from a tumor cell.

According to some embodiments, the expression is mRNA expression or protein expression.

According to some embodiments, the SETDB1 expression is cytoplasmic SETDB1 expression.

According to some embodiments, the method is an in vitro method.

According to some embodiments, the method further comprises administering the MTA to a suitable subject.

According to some embodiments, the MTA is a microtubule destabilizing agent and further comprising enhancing HDAC6 expression, function or both in the suitable subject.

According to some embodiments, the MTA is a microtubule stabilizing agent and further comprising decreasing HDAC6 expression, function or both in the suitable subject.

According to some embodiments, the HDAC6 expression or function is within a cancer cell.

According to another aspect, there is provided a method of treating a subject suffering from a cancer characterized by increased SETDB1 expression, the method comprising reducing microtubule stability in the cancer, thereby treating the subject.

According to some embodiments, the reducing comprises administering an MTA that destabilizes microtubules.

According to some embodiments, the reducing comprises increasing HDAC6 expression, function or both in the cancer.

According to another aspect, there is provided a method of treating a subject suffering from a cancer characterized by decreased or healthy SETDB1 expression, the method comprising increasing microtubule stability in the cancer, thereby treating the subject.

According to some embodiments, the increasing comprises administering an MTA that stabilizes microtubules.

According to some embodiments, the increasing comprises decreasing HDAC6, expression, function or both in the cancer.

According to some embodiments, the method further comprises measuring SETDB1 expression in the cancer.

According to another aspect, there is provided a kit comprising:
a. a microtubule targeting agent (MTA); and
b. an agent for specific detection of SETDB1 expression.

According to another aspect, there is provided a kit comprising an agent for specific detection of SETDB1 for use in a method of determining suitability of a subject in need thereof to be treated by a microtubule targeting agent (MTA).

According to some embodiments, the kit is for use in a method of determining suitability of a subject in need thereof to be treated by a microtubule targeting agent (MTA).

According to some embodiments, the method is a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-J: SETDB1 is found in the cytoplasm and associates with MTs. (1A) Western blot analysis of nuclear (Nuc) and cytoplasmic (Cyt) fractions of mouse B16-F1 cells and human WM266.4 cells for SETDB1, SUV39H2, α-Tubulin and histone H3. (1B-C) B16-F1 cells immunostained for (1B) SETDB1 or (1C) SUV39H2 and α-Tubulin. DNA stained with Hoechst 33342. Sections inside the inserts are magnified at the right side of each micrograph. The merged images show the merged signals of SETDB1, α-Tubulin and Hoechst. Scale bar: 50 μm. (1D-E) WM266.4 cells immunostained for SETDB1 with two different commercial antibodies (1D) SCB=Santa Cruz Biotechnology sc-66884, (1E) CST=Cell Signaling Technology 93212) and α-Tubulin. Sections inside the inserts are magnified at the right side of each micrograph. The merged images show the merged signals of SETDB1 and α-Tubulin. Scale bar: 20 μm. (1F) HeLa cells immunostained for SETDB1 and α-Tubulin. The merged images show the merged signals of SETDB1 and α-Tubulin. Scale bar: 20 μm. (1G) Mitotic B16-F1 cells and HeLa cells immunostained for SETDB1 and α-Tubulin. DNA stained with Hoechst 33342. Sections inside the inserts are magnified at the right side of each micrograph. The merged images show the merged signals of SETDB1, α-Tubulin and Hoechst. Scale bar: 5 μm. (1H-I) WM266.4 cells transfected with either (1H) pEGFP-Moesin or (1I) pEGFP-SETDB1 immunostained for GFP and α-Tubulin. Sections inside the inserts are magnified at the right side of each micrograph. The merged images show the merged signals of GFP and α-Tubulin. Scale bar: 20 μm. (1J) Co-immunoprecipitation of SETDB1 and α-Tubulin. Over-expressed GFP-fused SETDB1 was immunoprecipitated with anti GFP antibodies. Western blot showing staining with anti-SETDB1 and anti-α-Tubulin antibodies.

FIGS. 2A-C: SETDB1 KD enhances MT polymerization rate. (2A-B) MTs were disrupted in B16-F1 cells transfected with either (2A) Ctl siRNA or (2B) siRNA against SETDB1 by nocodazole treatment for 3 hours. Following nocodazole removal, cells were further incubated for the indicated time periods to allow MT polymerization. After fixation, cells were immunostained with antibodies against α-Tubulin, γ-Tubulin and DNA was stained with Hoechst 33342. The white arrows indicate the localization of the MTOCs. Scale bar: 20 μm. (2C) Quantification of the recovery rate of MTs after nocodazole washout. The area covered by MTs around the MTOC was quantified by ImageJ software and normalized to the area in cells transfected with ctl siRNA. The bar graph shows the average of the relative area covered by MTs of three repetitions ±SE. At least of 30 cells were measured for each condition in each repetition. Statistical significance was calculated with Student's t-test, **P<0.01.

FIGS. 3A-C: SETDB1 over-expression enhances MT polymerization rate. (3A-B) MTs were disrupted in B16-F1 cells transfected with either (3A) pEGFP-Moesin or (3B) pEGFP-SETDB1 by nocodazole treatment for 3 hours. Following nocodazole removal, cells were further incubated for 3 minutes to allow MT polymerization. After fixation, cells were immunostained with antibodies against GFP and α-Tubulin and DNA was stained with Hoechst 33342. The white arrows indicate the localization of the MTOCs. Scale bar: 20 μm. (3C) Quantification of the recovery rate of MTs after nocodazole washout. The area covered by MTs around the MTOC was quantified by ImageJ software and normalized to the area in cells transfected with pEGFP-Moesin. The bar graph shows the average of the relative area covered by MTs of three repetitions ±SE. At least of 30 cells were measured for each condition in each repetition. Statistical significance was calculated with Student's t-test, ** P<0.01.

FIG. 4A-E. SETDB1 KD affects MT dynamics. (4A) Micrographs showing signals of GFP comets in HeLa cells co-transfected with EB1-GFP vector and either Ctl siRNA or siRNA against SETDB1. Frames were taken every 3 seconds. Scale bar: 5 μm. (4B-E) Parameters of MT dynamics as measured by EB1 tracking in time lapse microscopy. The bar charts represent the ratios of the indicated parameters, (4B) Growth duration, (4C) Growth length, (4D) Growth rate, and (4E) Catastrophe frequency, of MTs in SETDB1 KD cells to Ctl cells. The averages are of three repetitions ±SE. In each experiment a minimum of 100 EB1-GFP comets per condition were measured. Statistical significance was calculated with Student's t-test, * P<0.05.

FIGS. 5A-H: SETDB1 KD alters the duration of mitotic phases. (5A) Relative proliferation rate of SETDB1 KD HeLa cells as measured by the XTT assay. The average of three repetitions ±SE is shown. Statistical significance was calculated with Student's t-test, * P<0.05. (5B) The percentage of successful and failed mitotic event in HeLa cells transfected with either Ctl siRNA or siRNA against SETDB1. Statistical significance was calculated with Student's t-test, ** P<0.01. (5C) Micrographs showing mitotic progression of HeLa cells transfected with either Ctl siRNA or siRNA against SETDB1. Scale bar: 25 μm. (5D-H) Time periods of the indicated phases during cell division, (5D) NEB to anaphase, (5E) anaphase to cleavage furrow, (5F) NEB to cleavage furrow, (5G) cleavage furrow to abscission, and (5H) NEB to ICB abscission, as calculated from time-lapse images of HeLa cells transfected with either Ctl siRNA or siRNA against SETDB1. Frames were taken every 3 minutes. In each experiment a minimum of 40 cells were tracked for each condition. The average values of a representative experiment out of three experiments are presented. Statistical significance was calculated with Wilcoxon-Mann-Whitney test, ** P<0.01.

FIGS. 6A-D: SETDB1 over-expression enhances MT polymerization rate in KMT-independent manner. (6A-C) MTs were disrupted in B16-F1 cells transfected with either (6A) pEGFP-Moesin, (6B) pEGFP-SETDB1 WT or (6C) pEGFP-SETDB1 CD by nocodazole treatment for 3 hours. Following nocodazole removal, cells were further incubated for 3 minutes to allow MT polymerization. After fixation, cells were immunostained with antibodies against GFP and α-Tubulin and DNA was stained with Hoechst 33342. The white arrows indicate the localization of the MTOCs. Scale bar: 20 μm. (6D) Quantification of the recovery rate of MTs after nocodazole washout. The area covered by MTs around the MTOC was quantified by ImageJ software and normalized to the area in cells transfected with pEGFP-Moesin. The bar graph shows the average of the relative area covered by MTs of three repetitions ±SE. At least of 30 cells were measured for each condition in each repetition. Statistical significance was calculated with Student's t-test, * P<0.05, **P<0.01.

FIGS. 7A-C: SETDB1-HDAC6 interaction. (7A) Co-IP of over-expressed SETDB1-GFP and HDAC6-FLAG in 293 cells. (7B) Western blot analysis of the Ac-Tubulin levels in B16-F1 cells transfected with either Ctl siRNA or siRNA against SETDB1. (7C) Bar graph of the ratio of Ac-Tubulin to Tubulin levels in three repetitions normalized to the same ratio in Ctl siRNA-transfected cells +/−SE. Statistical significance was calculated with Student's t-test, * P<0.05.

### DETAILED DESCRIPTION OF THE INVENTION

The present invention, in some embodiments, provides methods determining suitability of a subject to be treated with a microtubule targeting agent. The present invention further concerns a method of treating cancer with a microtubule targeting agent. Kits comprising agents for specific detection of SETDB1 are also provided.

The invention is based on the surprising finding that SETDB1 has a specific cytoplasmic activity that promotes cancer formation and progression. Specifically, cytoplasmic SETDB1 co-localizes with the microtubule (MT) network in the cytoplasm. SETDB1 knockdown increased MT stability as measured by EB1-tracking and MT recovery from nocodazole treatment and interfered with mitotic progression. Over-expression of either wild type or catalytic dead (CD) SETDB1 increased MT polymerization rate to the same extent, suggesting SETDB1 affects MT dynamics in a methyltransferase-independent manner. Finally, there was found an interaction between SETDB1 and the tubulin deacetylase HDAC6 along with increased tubulin acetylation levels after knockdown of SETDB1, suggesting that SETDB1 can affect MT dynamics by supporting HDAC6 activity.

Microtubules must be dynamic in order for them to function properly. Too stable microtubules cannot be depolymerized and rearranged. Microtubules with very short half-life (too high frequency of catastrophe events) will fail to generate the required network. Especially during mitosis, a fully dynamic network is essential. For this reason, a cancer cell with low SETDB1 levels, which has a strengthened network, may be targeted by therapeutics that further stabilize the microtubules. This could produce cells with very strong and non-dynamic microtubules which is fatal during mitosis. Conversely, a cancer cell with high SETDB1 levels, which has a weakened network, may be targeted by therapeutic that further destabilize the microtubules. This could produce cells with very weak microtubules that are prone to catastrophe especially during mitosis. Beyond this, SETDB1 levels strongly indicate what therapeutics not to administer. A cancer with low SETDB1 levels has a strengthened network and thus is unlikely to be susceptible to destabilizing drugs. Similarly, a cancer with high SETDB1 levels and a weakened network is unlikely to be susceptible to strengthening drugs. Thus, when the SETDB1 status is known in a patient, a personalized selection of microtubule targeting agents (MTA) can be made.

By a first aspect, there is provided a method of determining suitability of a subject to be treated with a microtubule targeting agent (MTA), the method comprising measuring SETDB1 expression in the subject and determining suitability based on the SETDB1 expression, thereby determining suitability of the subject to be with the MTA.

In some embodiments, the method is a diagnostic method. In some embodiments, the method is a prognostic method. In some embodiments, the method is a method of determining a personalized treatment regimen. In some embodiments, the method is an ex vivo method. In some embodiments, the method is an in vitro method. In some embodiments, the method is an in vivo method. In some embodiments, the method comprises receiving a sample from the subject. In some embodiments, the method comprises providing a sample from the subject. In some embodiments, the method comprises extracting a sample from the subject. In some embodiments, the measuring is measuring within the sample. In some embodiments, expression is expression within the sample. In some embodiments, expression is expression in the subject. In some embodiments, expression is expression in a disease cell. In some embodiments, expression is expression in a cell to be treated by the MTA. In some embodiments, the cell is a cancer cell.

In some embodiments, the sample is a biological sample. In some embodiments, the sample is a tumor sample. In some embodiments, the sample is a biopsy. In some embodiments, the sample comprises cells. In some embodiments, the sample comprises cancer cells. In some embodiments, the sample is a primary sample. In some embodiments, the sample is a sample in culture. In some embodiments, the sample is from the subject. In some embodiments, the sample comprises DNA from a cancer cell. In some embodiments, the DNA is cell free DNA (cfDNA). In some embodiments, the DNA comprises at least a portion of the SETDB1 genomic locus. In some embodiments, the sample comprises cancer cell RNA. In some embodiments, the RNA is mRNA. In some embodiments, the sample comprises cancer cell protein.

In some embodiments, the subject is a mammal. In some embodiments, the subject is a human. In some embodiments, the subject is in need of a method of the invention. In some embodiments, the subject is in need to treatment by an MTA. In some embodiments, the subject suffers from a disease, disorder or condition. In some embodiments, the disease, disorder or condition is treatable by the MTA. In some embodiments, the disease is a proliferative disease. In some embodiments, the disease is characterized by cellular replication. In some embodiments, cellular replication is aberrant cellular replication. In some embodiments, the proliferative disease is cancer. In some embodiments, the cancer is a cancer treatable with MTAs. In some embodiments, the cancer is a cancer treatable by the MTA. In some embodiments, the cancer is known to express SETDB1. In some embodiments, express is over-express. In some embodiments, the cancer is characterized by SETDB1 expression. In some embodiments, expression is over-expression. In some embodiments, over-expression is beyond a predetermined threshold. In some embodiments, over-expression is expression higher than the expression in a healthy subject. In some embodiments, over-expression is expression higher than the expression in a healthy sample. In some embodiments, the cancer is selected from brain, skin, breast, lung, renal, liver, pancreatic, head and neck, hematopoietic, endometrial, bladder, sarcoma, glioma, colorectal, gastric, prostate, ovarian, testicular, and cervical cancer. In some embodiments, the cancer is selected from skin, breast, lung, ovarian, and cervical cancer. In some embodiments, the cancer is a cancer treatable with an MTA. In some embodiments, the skin cancer is melanoma. In some embodiments, the cancer is a solid cancer. In some embodiments, the cancer is a tumor. In some embodiments, the cancer is a hematopoietic cancer. In some embodiments, the hematopoietic cancer is selected from leukemia and lymphoma.

In some embodiments, the expression is mRNA expression. In some embodiments, the expression is protein expression. In some embodiments, the expression is mRNA or protein expression. In some embodiments, expression is expression of SETDB1. In some embodiments, expression is cytoplasmic expression. In some embodiments, determining expression is determining the presence of a gene duplication of SETDB1. In some embodiments, a gene duplication of SETDB1 indicates overexpression of SETDB1.

The term “expression” as used herein refers to the biosynthesis of a gene product, including the transcription and/or translation of the gene product. Thus, expression of a nucleic acid molecule may refer to transcription of the nucleic acid fragment (e.g., transcription resulting in mRNA or other functional RNA) and/or translation of RNA into a precursor or mature protein (polypeptide). Thus, either mRNA or protein can be measured to determine expression. In some embodiments, expression is expression level. Methods of determining gene expression are well known in the art and any such method may be employed.

A variety of known techniques may be suitable for determining expression. Such techniques include methods based on hybridization analysis of polynucleotides and on sequencing of polynucleotides, and proteomics-based methods. In some embodiments, the measuring step is performed by nucleic acid hybridization, nucleic acid amplification, or an immunological method. In some embodiments, the measuring step is performed in-situ. In some embodiments, fluorescence labeling or staining are applied. In some embodiment, an imaging step is further applied.

In some embodiments, the expression is obtained by measuring protein levels of SETDB1. In some embodiments, the expression, and the level of expression, of proteins or polypeptides can be detected through immunohistochemical staining of tissue slices or sections. In some embodiments, the tissue is tumor tissue. Additionally, SETDB1 proteins/polypeptides can be detected by Western blotting, ELISA or Radioimmunoassay (RIA) assays employing protein-specific antibodies. In some embodiments, the measuring comprises extracting proteins from the sample. In some embodiments, the measuring comprises lysing the sample. In some embodiments, protein is extracted from the lysate. Lysing buffers and protein sample buffers, i.e., laemmli buffer, STM buffer, TP buffer, SDS sample buffer and the like, are well known in the art and any suitable buffer may be used. In some embodiments, measuring is by western blot. In some embodiments, the measuring is by antibody-based detection. Anti-SETDB1 antibodies are well known in the art and examples of such are provided hereinbelow. Any such antibody may be used for SETDB1 protein detection. In some embodiments, the antibody is rabbit anti-SETDB1 (Santa Cruz Biotechnology sc-66884). In some embodiments, the antibody is anti-SETDB1 (Cell Signaling Technology, 93212).

Alternatively, SETDB1 protein levels can be determined by constructing or using an antibody microarray in which binding sites comprise immobilized, preferably monoclonal, antibodies specific to a plurality of proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art.

In some embodiments, the determining step comprises the step of obtaining nucleic acid molecules from the sample. In some embodiments, nucleic acids are obtained from the lysate. In some embodiments, obtaining is isolating. In some embodiments, the nucleic acids molecules are selected from mRNA molecules, DNA molecules and cDNA molecules. In some embodiments, the cDNA molecules are obtained by reverse transcribing the mRNA molecules. In some embodiments, the expression is determined by measuring mRNA levels of SETDB1. Methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995).

Numerous methods are known in the art for measuring expression levels of one or more genes such as by amplification of nucleic acids (e.g., PCR, isothermal methods, rolling circle methods, etc.) or by quantitative in situ hybridization. Design of primers for amplification of specific genes is well known in the art, and such primers can be found or designed on various websites such as bioinfo.ut.ee/primer3-0.4.0/ or pga.mgh.harvard.edu/primerbank/ for example. In some embodiments, the primers are the primers provided hereinbelow. In some embodiments, the primers are selected from fragments of SEQ ID NO: 1-4. In some embodiments, the primers are ATGTCTTCCCTTCCTGGGTGCAT (SEQ ID NO: 5) and CTAAAGAAGACGTCCTCTGCATTCAAT (SEQ ID NO: 6).

The skilled artisan will understand that these methods may be used alone or combined. Non-limiting exemplary method are described herein.

RT-qPCR: A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR (qPCR) produces a measurement of an increase or decrease in copies of the original RNA and has been used to attempt to define changes of gene expression in cancer tissue as compared to comparable healthy tissues. In some embodiments, the PCR is qPCR.

RNA-Seq: RNA-Seq uses recently developed deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq. Following sequencing, the resulting reads are either aligned to a reference genome or reference transcripts or assembled de novo without the genomic sequence to produce a genome-scale transcription map that consists of both the transcriptional structure and/or level of expression for each gene. To avoid artifacts and biases generated by reverse transcription direct RNA sequencing can also be applied. In some embodiments, the sequencing is next-generation sequencing. In some embodiments, the sequencing is deep sequencing. In some embodiments, the sequencing is shotgun sequencing.

Microarray: Expression levels of a gene may be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from RNA of a test sample. As in the RT-PCR method, the source of RNA typically is total RNA isolated from a tumor sample, and optionally from normal tissue of the same patient as an internal control or cell lines. RNA can be extracted, for example, from frozen or archived paraffin-embedded and fixed (e.g., formalin-fixed) tissue samples. For archived, formalin-fixed tissue cDNA-mediated annealing, selection, extension, and ligation, DASL-Illumina method may be used. For a non-limiting example, PCR amplified cDNAs to be assayed are applied to a substrate in a dense array. Microarray analysis can be performed by commercially available equipment, following manufacturer's protocols, such as by using the Affymetrix GenChip technology, or Incyte's microarray technology.

As used herein, “SETDB1” refers to SET domain bifurcated histone lysine methyltransferase 1. SETDB1 is also known as KMT1E, ESET, H3-K9-HMTase 4, KG1T, TDRD21 and H3K9 methyltransferase ESET. SETDB1 is best known as a histone methyltransferase that primarily methylates H3 at lysine 9 (K9). It can mono, di or tri methylate K9 which is known to be associated with silent chromatin (primarily me2 and me3). SETDB1 is best known for its nuclear functions but is also known to have cytoplasmic expression in some cells. Cancers with overexpression of SETDB1 are known to have cytoplasmic SETDB1 and indeed SETDB1 overexpression is known to result in primarily cytoplasmic overexpression. In some embodiments, SETDB1 expression is total SETDB1 expression. In some embodiments, total expression is cytoplasmic and nuclear expression. In some embodiments, SETDB1 expression is SETDB1 cytoplasmic expression. In some embodiments, SETDB1 is cytoplasmic SETDB1. In some embodiments, cytoplasmic SETDB1 is SETDB1 with cytoplasmic localization.

In some embodiments, SETDB1 is mammalian SETDB1. In some embodiments, SETDB1 is human SETDB1. In some embodiments, human SETDB1 comprises or consists of the nucleotide sequence provided in Entrez gene accession number 9869. In some embodiments, mouse SETDB1 comprises or consists of the nucleotide sequence provided in Entrez gene accession number 84505. In some embodiments, human SETDB1 comprises or consists of the amino acid sequence provided in UniProt number Q15047. In some embodiments, mouse SETDB1 comprises or consists of the amino acid sequence provided in UniProt number 088974. In some embodiments, human SETDB1 mRNA comprises or consists of the nucleotide sequence selected from those provided in RefSeq number NM_001145415, NM 001243491, NM_012432, NM_001366417, and NM_001366418. In some embodiments, human SETDB1 mRNA comprises or consists of the nucleotide sequence provided in RefSeq number NM_001145415. In some embodiments, human SETDB1 mRNA comprises or consists of the nucleotide sequence provided in RefSeq number NM_001243491. In some embodiments, human SETDB1 mRNA comprises or consists of the nucleotide sequence provided in RefSeq number NM_012432. In some embodiments, human SETDB1 mRNA comprises or consists of the nucleotide sequence provided in RefSeq number NM_001366417. In some embodiments, human SETDB1 mRNA comprises or consists of the nucleotide sequence provided in RefSeq number NM_001366418. In some embodiments, mouse SETDB1 mRNA comprises or consists of the nucleotide sequence selected from those provided in RefSeq number NM_001163641, NM_001163642 and NM_018877. In some embodiments, mouse SETDB1 mRNA comprises or consists of the nucleotide sequence provided in RefSeq number NM_001163641. In some embodiments, mouse SETDB1 mRNA comprises or consists of the nucleotide sequence provided in RefSeq number NM_001163642. In some embodiments, mouse SETDB1 mRNA comprises or consists of the nucleotide sequence provided in RefSeq number NM_018877. In some embodiments, human SETDB1 protein comprises or consists of the amino acid sequence selected from those provided in RefSeq number NP_001138887, NP_001230420, NP_036564, NP_001353346, and NP_001353347. In some embodiments, human SETDB1 protein comprises or consists of the amino acid sequence provided in RefSeq number NP_001138887. In some embodiments, human SETDB1 protein comprises or consists of the amino acid sequence provided in RefSeq number NP_001230420. In some embodiments, human SETDB1 protein comprises or consists of the amino acid sequence provided in RefSeq number NP_036564. In some embodiments, human SETDB1 protein comprises or consists of the amino acid sequence provided in RefSeq number NP_001353346. In some embodiments, human SETDB1 protein comprises or consists of the amino acid sequence provided in RefSeq number NP_001353347.

As used herein, the terms “microtubule” and “MT” refer to at least one polymer of tubulin. In some embodiments, a microtubule is a polymer of cytoskeletal tubulin. Microtubules are highly dynamic polymers that grow and shrink with the cells needs. It forms that backbone of the cell and specifically the cytoskeleton and allow for both retrograde and anterograde transport. Microtubules have an outer diameter of between 23 and 27 nanometers (nm) and an inner diameter of between 11 and 15 nm.

As used herein, the terms “microtubule targeting agent” and “MTA” refer to agents that bind to tubulin or/and microtubules and affect their properties and function. In some embodiments, a microtubule property is microtubule dynamics. In some embodiments, microtubule function is microtubule polymerization. In some embodiments, microtubule function is microtubule depolymerization. In some embodiments, microtubule function is cargo transport. In some embodiments, cargo transport comprises motor protein activity. In some embodiments, the cargo is selected from proteins, RNAs, vesicles and organelles. It will be understood by a skilled artisan that the microtubule network is responsible for all variety of cargo transport throughout the cell. In some embodiments, the agents are small molecule agents. In some embodiments, the agents are proteinaceous agents. In some embodiments, the agents directly bind to microtubules. In some embodiments, the agents bind to tubulin. In some embodiments, tubulin is beta tubulin. In some embodiments, the tubulin is alpha tubulin. In some the MTA is a microtubule stabilizing agent. In some embodiments, the MTA is a microtubule destabilizing agent. In some embodiments, a destabilizing agent is a disrupting agent. In some embodiments, the MTA is selected from a microtubule stabilizing agent and a microtubule disrupting agent.

As used herein, the term “stabilizing agent” refers to an agent that stabilizes a tubulin polymer. In some embodiments, stabilizing comprises strengthening. In some embodiments, stabilizing comprises reducing polymer degradation. In some embodiments, stabilizing comprises reducing tubulin monomer removal from the polymer. In some embodiments, stabilizing comprising increasing the rate of tubulin polymerization. In some embodiments, stabilizing comprises increasing tubulin monomer addition. In some embodiments, stronger microtubules result in longer mitosis. In some embodiments, stronger microtubules result in attenuated proliferation. In some embodiments, stronger microtubules inhibit mitotic progression. In some embodiments, increase is an increase of at least 10, 20, 25, 30, 40, 50, 60, 70, 75, 80, 90 or 100%. Each possibility represents a separate embodiment of the invention. In some embodiments, decrease is a decrease of at least 10, 20, 25, 30, 40, 50, 60, 70, 75, 80, 90 or 100%. Each possibility represents a separate embodiment of the invention. In some embodiments, increased/decreased is as compared to a healthy control. In some embodiments, increased/decreased is as compared to expression in a healthy control. In some embodiments, increased/decreased is as compared to stability in a healthy control. In some embodiments, the healthy control is a healthy cell. In some embodiments, a healthy cell is a non-cancerous cell.

In some embodiments, the MTA is an MT stabilizing agent and expression of SETDB1 at or below a predetermined threshold indicates the subject is suitable for treatment with the MTA. In some embodiments, the MTA is an MT stabilizing agent and expression of SETDB1 above a predetermined threshold indicates the subject is unsuitable for treatment with the MTA. In some embodiments, the MTA is an MT stabilizing agent and expression of SETDB1 above healthy SETDB1 levels indicates the subject is unsuitable for treatment with the MTA. In some embodiments, the MTA is an MT stabilizing agent and expression of SETDB1 at or below healthy SETDB1 levels indicates the subject is suitable for treatment with the MTA. In some embodiments, healthy SETDB1 levels are SETDB1 levels in a healthy cell. In some embodiments, the healthy cell is of the same cell type as the disease cell. In some embodiments, the healthy cell is of the same cell type as the cancer cell.

In some embodiments, the MTA is an MT stabilizing agent and expression of SETDB1 at or below a predetermined threshold indicates the subject is unsuitable for treatment with the MTA. In some embodiments, the MTA is an MT stabilizing agent and expression of SETDB1 above a predetermined threshold indicates the subject is suitable for treatment with the MTA. In some embodiments, the MTA is an MT stabilizing agent and expression of SETDB1 above healthy SETDB1 levels indicates the subject is suitable for treatment with the MTA. In some embodiments, the MTA is an MT stabilizing agent and expression of SETDB1 at or below healthy SETDB1 levels indicates the subject is unsuitable for treatment with the MTA.

In some embodiments, the stabilizing agent is a taxane. As used herein, “taxanes” refer to a class of diterpenes comprising (1S,3R,4R,8S,11S,12R)-4,8,12,15,15-pentamethyltricyclo[9.3.1.03,8]pentadecane (CAS number 1605-68-1). In some embodiments, the taxane is selected from paclitaxel, docetaxel, larotaxel tesetaxel, TPI-287, cabazitaxel, 10-deacetylbaccatin III, baccatin III, and 7-epipaclitaxe and derivatives thereof. In some embodiments, the taxane is selected from paclitaxel, docetaxel, cabazitaxel, 10-deacetylbaccatin III, baccatin III, and 7-epipaclitaxe. In some embodiments, the taxane is paclitaxel. In some embodiments, a paclitaxel derivative is selected from paclitaxel C, docetaxel, cabazitaxel, and Abraxane. Methods of making TMA derivatives are well known in the art. For example, Cao et al., “Recent advances in microtubule-stabilizing agents”, European Journal of Medicinal Chemistry, 2018 Jan. 1; 143:806-828, Borys et al., “Intrinsic and extrinsic factors affecting microtubule dynamics in normal and cancer cells”, Molecules, 2020 August; 25(16): 3705 and Zhang and Kanakkanthara, “Beyond the Paclitaxel and Vinca Alkaloids: next generation of plant-derived microtubule-targeting agents with potential anticancer activity”, Cancers (Basel), 2020 July; 12(7): 1721, which are all hereby incorporated by reference in their entirety, provide numerous examples of TMAs as well as well as methods of generating TMA derivatives. In some embodiments, the taxane is conjugated to a half-life extending moiety. Abraxane is an example of such a conjugate. Any moiety that extends the half-life of the taxane may be used. In some embodiments, a derivative is a derivative that stabilizes microtubules.

In some embodiments, the MT stabilizing agent binds tubulin in the paclitaxel binding site. In some embodiments, the MT stabilizing agent binds tubulin outside of the paclitaxel binding site. In some embodiments, the MT destabilizing agent binds tubulin in the vinca binding site. In some embodiments, the MT destabilizing agent binds tubulin outside of the vinca binding site. In some embodiments, the MTA is a naturally occurring molecule. In some embodiments, the naturally occurring molecule is a bacterial molecule. In some embodiments, the naturally occurring molecule is a plant molecule. In some embodiments, the naturally occurring molecule is a marine organism molecule. In some embodiments, the MTA is a small molecule. In some embodiments, the small molecule is synthetic.

In some embodiments, the MT stabilizing agent is a taccalonolide. Taccalonolides are highly oxygenated pentacyclic steroids isolated from the plant Tacca plantaginea Andre. In some embodiments, the taccalonlide is selected from taccalonolide A, taccalonolide B, taccalonolide E and taccalonolide N. In some embodiments, the taccalonlide is selected from taccalonolide A, taccalonolide B, taccalonolide E, taccalonolide J and taccalonolide N. In some embodiments, the taccalonlide is taccalonolide A. In some embodiments, the taccalonlide is taccalonolide B. In some embodiments, the taccalonlide is taccalonolide E.

In some embodiments, the taccalonlide is taccalonolide J. In some embodiments, the taccalonlide is taccalonolide N. In some embodiments, the taccalonolide is a taccalonolide derivative. In some embodiments, the taccalonolide derivative is selected from taccalonolide Z, AA, AB, T, R, AO, AC, I, AD, H2, AF, AJ, AK, AL, AM and AN.

In some embodiments, the MT stabilizing agent is an epothilone. Epothilones are bacterial macrolides isolated from Sorangium cellulosum. In some embodiments, the epothilone is selected from epothilone A, epothilone B, epothilone C, epothilone D, epothilone E, and epothilone F. In some embodiments, the epothilone is epothilone A. In some embodiments, the epothilone is epothilone B. In some embodiments, the epothilone is epothilone C. In some embodiments, the epothilone is epothilone D. In some embodiments, the epothilone is epothilone E. In some embodiments, the epothilone is an epothilone derivative. In some embodiments, the epothilone derivative is ixabepilone (BMS-247550).

In some embodiments, the epothilone derivative is sagopilone. In some embodiments, the epothilone derivative is patupilone. In some embodiments, the epothilone derivative is BMS-310705. In some embodiments, the epothilone derivative is epothilone D. In some embodiments, the epothilone derivative is KOS-1584. In some embodiments, the epothilone derivative is BMS-753493.

In some embodiments, the MT stabilizing agent is a ceratamine. Ceratamines are heterocyclic alkaloids with an atypical imidazo [4, 5, d] azepine core heterocycle. In some embodiments, the ceratamine is selected from ceratamine A and ceratamine B. In some embodiments, the ceratamine is a ceratamine derivative.

In some embodiments, the MT stabilizing agent is FR182877. In some embodiments, FR182877 is WS9885B. In some embodiments, FR182877 is cyclostreptin. FR182877 is a bacterial metabolic product from Streptomyces Sp. No.9885. In some embodiments, the MT stabilizing agent is dictyostatin. In some embodiments, the MT stabilizing agent is a dictyostatin derivative. In some embodiments, a derivative is a disatereomer. In some embodiments, the MT stabilizing agent is discodermolide. In some embodiments, MT stabilizing agent is a discodermolide derivative. In some embodiments, the discodermolide derivative is a discodermolide-paclitaxel hybrid. In some embodiments, the discodermolide-paclitaxel hybrid is KS-1-199-32. In some embodiments, the MT stabilizing agent is eleutherobin. In some embodiments, the MT stabilizing agent is a derivative of eleutherobin. In some embodiments, the MT stabilizing agent is sarcodictyin A. In some embodiments, the MT stabilizing agent is sarcodictyin B. In some embodiments, the MT stabilizing agent is zampanolide. In some embodiments, the MT stabilizing agent is dactylolide. In some embodiments, the MT stabilizing agent is laulimalide. In some embodiments, the MT stabilizing agent is isolaulimalide. In some embodiments, the MT stabilizing agent is a derivative of laulimalide. In some embodiments, the derivative of laulimalide is isolaulimalide. In some embodiments, the MT stabilizing agent is peloruside A. In some embodiments, the MT stabilizing agent is persin. In some embodiments, the MT stabilizing agent is a persin analog. In some embodiments, the MT stabilizing agent is a derivative of persin.

In some embodiments, the MT stabilizing agent is GS-164. In some embodiments, the MT stabilizing agent is Synstab A. In some embodiments, the MT stabilizing agent is 4′-methoxy-2-styrylchromone. In some embodiments, the MT stabilizing agent is a dienone derivative. In some embodiments, the MT stabilizing agent is a (Z)-1-aryl-3-arylamino-2-propen-1-one. In some embodiments, the a (Z)-1-aryl-3-arylamino-2-propen-1-one is (Z)-1-(2-bromo-3, 4, 5-trimethoxyphenyl)-3-(3-hydroxy-4-methoxyphenylamino)prop-2-en-1-one. In some embodiments, the MT stabilizing agent is a pyranochalcone derivative. In some embodiments, the MT stabilizing agent is an a-cyano bis(indolyl)chalcone. In some embodiments, the MT stabilizing agent is a cyclopropylamide analog of combretastatin-A4.

In some embodiments, the MT stabilizing agent is an HDAC6 inhibitor. HDAC6 is histone deacetylase 6 and any known inhibitor may be employed. In some embodiments, the inhibitor inhibits HDAC6 expression. In some embodiments, the inhibitor inhibits HDAC6 function. In some embodiments, the inhibitor is an inhibitory nucleic acid molecule. In some embodiments, the inhibitory nucleic acid molecule inhibits translation of an HDAC6 mRNA. In some embodiments, the inhibitory nucleic acid molecule degrades an HDAC6 mRNA. In some embodiments, the inhibitory nucleic acid molecule is an antisense oligonucleotide (ASO). In some embodiments, the inhibitory nucleic acid molecule is selected from an siRNA, an shRNA, and a miRNA. In some embodiments, the inhibitor is a small molecule inhibitor. HDAC inhibitors are well known in the art and any such may be employed. In some embodiments, the inhibitor is a pan-HDAC inhibitor. In some embodiments, the inhibitor is an HDAC6-specific inhibitor. In some embodiments, the inhibitor inhibits a subclass of HDACs that includes HDAC6. Examples of HDAC6-specific inhibitors include, but are not limited to Tubacin, MPT0G413, SC-223877, Tubastatin, Tubastatin A hydrochloride, BSANP@JOC1, and BATCP. Examples of pan-HDAC inhibitors include, but are not limited to verinostat, Tricostatin A, and M344. Examples of inhibitors that inhibit HDAC6 and other HDACs include, but are not limited to, Tubastatin A, MC1568, 1-Naphthohydroxamic acid, Droxinostat, KD5170, and PCI-24781.

As used herein, the term “destabilizing agent” refers to an agent that stabilizes free tubulin monomers or dimers or disrupts a tubulin polymer. In some embodiments, destabilizing comprises weakening. In some embodiments, destabilizing comprises increasing polymer degradation. In some embodiments, destabilizing comprises increasing tubulin monomer removal from the polymer. In some embodiments, destabilizing comprising decreasing the rate of tubulin polymerization. In some embodiments, destabilizing comprises decreasing tubulin monomer addition. In some embodiments, weaker microtubules result in shorter mitosis. In some embodiments, weaker microtubules result in increased proliferation. In some embodiments, weaker microtubules promote mitotic progression.

In some embodiments, the MTA is an MT destabilizing agent and expression of SETDB1 at or below a predetermined threshold indicates the subject is unsuitable for treatment with the MTA. In some embodiments, the MTA is an MT destabilizing agent and expression of SETDB1 above a predetermined threshold indicates the subject is suitable for treatment with the MTA. In some embodiments, the MTA is an MT destabilizing agent and expression of SETDB1 above healthy SETDB1 levels indicates the subject is suitable for treatment with the MTA. In some embodiments, the MTA is an MT destabilizing agent and expression of SETDB1 at or below healthy SETDB1 levels indicates the subject is unsuitable for treatment with the MTA.

In some embodiments, the MTA is an MT destabilizing agent and expression of SETDB1 at or below a predetermined threshold indicates the subject is suitable for treatment with the MTA. In some embodiments, the MTA is an MT destabilizing agent and expression of SETDB1 above a predetermined threshold indicates the subject is unsuitable for treatment with the MTA. In some embodiments, the MTA is an MT destabilizing agent and expression of SETDB1 above healthy SETDB1 levels indicates the subject is unsuitable for treatment with the MTA. In some embodiments, the MTA is an MT destabilizing agent and expression of SETDB1 at or below healthy SETDB1 levels indicates the subject is suitable for treatment with the MTA.

In some embodiments, the MT destabilizing agent is a vinca alkaloid. As used herein, “vinca alkaloid” refers to a group of indole-indoline dimers which are alkaloids obtained from the vinca genus of plants. In some embodiments, the vinca alkaloid is selected from vinblastine, vincristine, colcemid, nocodazole, vindesine, vinorelbine, vincaminol, vineridine, vinburnine, vinpocetine, vinflunine, minovincine, methoxyminovincine, minovincinine, vincdifformine, desoxyvincaminol crypthophycin1, romidepsin (FK-228), halichondrin B, erilubin, soblidotin, dolastin 15, dolastin 10, and vincamajine. In some embodiments, the vinca alkaloid is selected from nocodazole and vinblastine. In some embodiments, the vinca alkaloid is nocodazole. In some embodiments, the vinca alkaloid is vinblastine.

In some embodiments, MT destabilizing agent is a maytansine. In some embodiments, MT destabilizing agent is a derivative of a maytansine. Maytansines are maytansinoids, macrolides of the ansamycin type from Maytenus ovatus. In some embodiments, MT destabilizing agent is maytansine. In some embodiments, the MT destabilizing agent is a macrocyclic polyketide. In some embodiments, the macrocyclic polyketide is disorazole Z. In some embodiments, the MT destabilizing agent is a lactone. In some embodiments, the lactone is a macrocyclic lactone. In some embodiments, the lactone is plocabulin (PM060184). In some embodiments, the lactone is emtansine. In some embodiments, the macrocyclic lactone is rhizoxin. In some embodiments, macrocyclic lactone is a macrocyclic lactone polyether. In some embodiments, the macrocyclic lactone polyether is spongistatin. In some embodiments, the MT destabilizing agent is curcumin. In some embodiments, the MT destabilizing agent is a derivative of circumin. In some embodiments, the derivative of circumin is selected from dimethylcurcumin, 3,5-dimethoxycurcumin, trimethoxycurcumin and Cl. In some embodiments, the derivative of circumin is selected from a ferrocenyl curcumin, pyrazole curcumin, isoxazole curcumin and benzylidiene curcumin derivatives.

In some embodiments, MT destabilizing agent is colchicine. In some embodiments, the MT destabilizing agent is a derivative of colchicine. In some embodiments, the MT destabilizing agent is combretastatin. In some embodiments, the MT destabilizing agent is a combretastatin derivative. In some embodiments, the combretastatin derivative is selected from combretastatin A-1, A-4, fosbretabulin, Oxi4503, combretastatin 4-O-phosphate (CA-4P), and ombrabulin. In some embodiments, the MT destabilizing agent is combretastatin A-1. In some embodiments, the MT destabilizing agent is combretastatin A-4. In some embodiments, the MT destabilizing agent is a combretastatin A-4 derivative. In some embodiments, the combretastatin A-4 derivative is ombrabulin. In some embodiments, the combretastatin A-4 derivative is CA-4P. In some embodiments, the combretastatin A-4 derivative is a cyclopropylamide analog of combretastatin-A4. In some embodiments, the derivative is a derivative that destabilizes microtubules.

In some embodiments, the MT destabilizing agent is TH588. In some embodiments, the MT destabilizing agent is a chalcone. In some embodiments, the chalcone is MDL27048. In some embodiments, the MT destabilizing agent is podophyllotoxin. In some embodiments, the MT destabilizing agent is a derivative of indole. In some embodiments, the derivative of indole is indibulin. In some embodiments, the MT destabilizing agent is a natural metabolite of estradiol. In some embodiments, the natural metabolite of estradiol is 2-methoxyestradiol. In some embodiments, the MT destabilizing agent is pironetin. In some embodiments, the MT destabilizing agent is a derivative of pironetin. In some embodiments, the MT destabilizing agent is a noscapinoid. In some embodiments, the MT destabilizing agent is noscapine. In some embodiments, the noscapinoid is noscapine. In some embodiments, the MT destabilizing agent is 9-bromonoscapine. In some embodiments, the noscapinoid is 9-bromonoscapine. In some embodiments, the MT destabilizing agent is quercetin.

In some embodiments, the MT destabilizing agent is HDAC6 or a homolog, derivative or fragment thereof. In some embodiments, the HDAC6 is human HDAC6. In some embodiments, the HDAC6 is mammalian HDAC6. In some embodiments, the HDAC6 is a homolog of HDAC6. In some embodiments, the HDAC6 is a derivative of HDAC6. In some embodiments, the derivative is HDAC6 or a homolog or fragment thereof conjugated to a half-life extending moiety. In some embodiments, the fragment is a functional fragment. In some embodiments, the function is deacetylation. In some embodiments, the deacetylation is deacetylation of tubulin. In some embodiments, the deacetylation is deacetylation of alpha tubulin. In some embodiments, the deacetylation of lysine 40 of alpha tubulin or its equivalent. In some embodiments, the deacetylation is lysine deacetylation. In some embodiments, the fragment comprises at least 10, 20, 25, 30, 40, 50, 60, 75, 80, 90 or 100 amino acids. Each possibility represents a separate embodiment of the invention.

As used herein, the term “half-life extending moiety” refers to a molecule or portion of a molecule that increases the half-life of an attached molecule. In some embodiments, attached is conjugated. In some embodiments, half-life is half-life in serum. In some embodiments, half-life is circulatory half-life. Half-life extending moieties are well known in the art and include, for example, albumen, IgG, antibody heavy chain and polyethylene glycol. Any known half-life extending moiety may be employed.

In some embodiments, the predetermined threshold is an expression in a healthy control. In some embodiments, the expression is an expression level. In some embodiments, expression is average expression. In some embodiments, the healthy control is a healthy sample. In some embodiments, the sample comprises cells. In some embodiments, the cells are healthy cells. In some embodiments, the healthy control is a healthy cell. In some embodiments, the control cell is of the same cell type as the disease cell. In some embodiments, the predetermined threshold is expression in a cell that does not comprise a duplication of the SETDB1 locus. In some embodiments, the cell is of the same cell type as the cell type of a disease cell. In some embodiments, the predetermined threshold is expression in a disease cell that does not comprise a duplication of the SETDB1 locus. In some embodiments, the predetermined threshold is expression in a disease cell that is not characterized by SETDB1 overexpression. In some embodiments, the disease cell is a cancer cell. In some embodiments, the predetermined threshold is expression in a disease cell that responds to treatment with an MTA. In some embodiments, the predetermined threshold is expression in a disease cell that does not responds to treatment with an MTA.

In some embodiments, the method further comprises administering the MTA. In some embodiments, the administering is to the subject. In some embodiments, the subject is a subject suitable for treatment. In some embodiments, the method is a method of diagnosis and treatment.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, intravenous, intramuscular, or intraperitoneal. In some embodiments, the administration is intratumoral administration.

The dosage administered will be dependent upon the age, health, and weight of the recipient, kind of concurrent treatment, if any, frequency of treatment, and the nature of the effect desired.

In some embodiments, the MTA is a microtubule destabilizing agent and the method further comprises enhancing HDAC6 expression, function or both in a suitable subject. In some embodiments, the method comprises enhancing HDAC6 expression. In some embodiments, the method comprises enhancing HDAC6 function. In some embodiments, the method comprises enhancing HDAC6 expression and function. In some embodiments, enhancing HDAC6 expression, function or both comprises administering HDAC6 or a homolog, derivative or fragment thereof.

In some embodiments, the MTA is a microtubule stabilizing agent and the method further comprises decreasing HDAC6 expression, function or both in a suitable subject. In some embodiments, the method comprises decreasing HDAC6 expression. In some embodiments, the method comprises decreasing HDAC6 function. In some embodiments, the method comprises decreasing HDAC6 expression and function. In some embodiments, decreasing HDAC6 expression, function or both comprises administering an HDAC6 inhibitor. In some embodiments, in a suitable subject is in a cell of the suitable subject. In some embodiments, the cell is a disease cell. In some embodiments, the disease cell is a cancer cell.

By another aspect, there is provided a method of treating a subject suffering from a cancer characterized by increased SETDB1 expression, the method comprising reducing microtubule stability in the cancer, thereby treating the subject.

By another aspect, there is provided a method of treating a subject suffering from a cancer characterized by decreased or healthy SETDB1 expression, the method comprising increasing microtubule stability in the cancer, thereby treating the subject.

By another aspect, there is provided an agent that reduces microtubule stability for use in treating a subject suffering from a cancer characterized by increased SETDB1 expression.

By another aspect, there is provided an agent that increases microtubule stability for use in treating a subject suffering from a cancer characterized by decreased or healthy SETDB1 expression.

In some embodiments, the cancer is characterized by abnormal SETDB1 expression. In some embodiments, the cancer is characterized by normal SETDB1 expression. In some embodiments, the abnormal is elevated. In some embodiments, abnormal is decreased. In some embodiments, the method further comprises measuring SETDB1 expression in the subject. In some embodiments, the method further comprises measuring SETDB1 expression in the cancer.

In some embodiments, the agent is an MTA. In some embodiments, an agent that reduces microtubule stability is an MTA that destabilized microtubules. In some embodiments, reducing comprises administering an MTA that destabilized microtubules. In some embodiments, reducing comprises administering a microtubule destabilizing MTA. In some embodiments, reducing comprises increasing HDAC6 expression, function or both. In some embodiments, the reducing is in the subject. In some embodiments, the reducing is in the cancer. In some embodiments, the reducing comprises administering HDAC6 or a homolog, derivative or fragment thereof.

In some embodiments, an agent that increases microtubule stability is an MTA that stabilizes microtubules. In some embodiments, increasing comprises administering an MTA that stabilizes microtubules. In some embodiments, increasing comprises administering a microtubule stabilizing MTA. In some embodiments, increasing comprises decreasing HDAC6 expression, function or both. In some embodiments, increasing is in the subject. In some embodiments, the increasing is in the cancer. In some embodiments, the increasing comprises administering an HDAC6 inhibitor.

By another aspect, there is provided a microtubule stabilizing MTA for use in treating a cancer characterized by decreased or healthy SETDB1 expression.

By another aspect, there is provided a microtubule destabilizing MTA for use in treating a cancer characterized by increased SETDB1 expression.

In some embodiments, increased is above a predetermined threshold. In some embodiments, increased is increased to above a predetermined threshold. In some embodiments, decreased is below a predetermined threshold. In some embodiments, decreased is decreased to below a predetermined threshold. In some embodiments, the threshold is the average expression in a cell. In some embodiments, the threshold is expression in a healthy cell. In some embodiments, the threshold is expression in a cell with a single copy of the SETDB1 locus. In some embodiments, the cell is a cell of the same cell type as the cell type of a diseased cell. In some embodiments, increased SETDB1 expression comprises cells with a duplication within the SETDB1 locus.

By another aspect, there is provide a kit comprising an agent for specific detecting of SETDB1 expression.

In some embodiments, the kit further comprises an MTA. In some embodiments, the kit further comprises instructions for performing a method of the invention. In some embodiments, the kit is for use in a method of determining suitability of a subject to be treated by an MTA. In some embodiments, the kit is for use in performing a method of the invention.

In some embodiments, an agent for specific detection of SETDB1 does not detect another protein or gene. In some embodiments, does not detect is does not significantly detect. In some embodiments, does not detect is does not detect at levels above background. In some embodiments, the agent is for detecting SETDB1 protein expression. In some embodiments, the agent comprises an anti-SETDB1 antibody. In some embodiments, the agent is an antibody array. In some embodiments, the agent is for detecting SETDB1 mRNA expression. In some embodiments, the agent is an SETDB1 primer. In some embodiments, the agent is an oligonucleotide complementary to a sequence of a SETDB1 mRNA. In some embodiments, complementary is reverse-complementary. In some embodiments, the agent is a microarray. In some embodiments, the kit is devoid of agents for detecting another protein or gene. In some embodiments, the kit comprises less than 10 detecting agents. In some embodiments, the kit comprises less than 20 detecting agents. In some embodiments, the kit comprises less than 100 detecting agents. In some embodiments, the kit comprises less than 10, 20, 25, 30, 40, 50, 60, 70, 75, 80, 90, 100, 150, 200, 250, 300, 350, 400, 450 or 500 detecting agents. Each possibility represents a separate embodiment of the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Cell culture: B16-F1 cells (ATCC, CRL-6323), WM266.4 cells, HeLa cells (ATCC, CCL-2) and 293 cells (ATCC, CRL-1573) were cultures in DMEM (Biological Industries, Kibbutz Beit-Haemek, Israel) supplemented with 10% fetal calf serum, 0.5% Penicillin-Streptomycin solution mix and 1% L-glutamine at 37° C. in a 7% CO₂ environment. Transfections of DNA plasmids were carried out with NanoJuice Transfection Kit (71900-3, Merck, Kenilworth, NJ, USA) according to the manufacturer's instructions. Cells were incubated for 24 hours before further analysis. For gene silencing cells were transfected with siRNA (IDT, Coralville, IA, USA) twice with a time interval of 48 hours using INTERFERin transfection reagent (Polyplus-transfection, Illkirch-Graffenstaden, France) according to the manufacturer's instructions. Cells were incubated for 24 hours after the second transfection before further analysis. SiRNAs used were mouse SETDB1(mm.Ri.Setdb1.13.1), human SETDB1 (hs.Ri.SETDB1.13.1) and negative control (51-01-14-04). Proliferation rate was measured with Cell Proliferation Kit (XTT based) (20-300-1000, Biological Industries, Kibbutz Beit-Haemek, Israel), according to manufacturer's protocol.

Plasmids and molecular cloning: Plasmids expressing GFP-fused WT and H1224K SETDB1 fused to GFP were generated by PCR using pREV-SETDB1 as a template. WT SETDB1 was amplified by KOD Hot Start DNA Polymerase (71086, Merck) and the oligonucleotides 5′-cagagctcATGTCTTCCCTTCCTGGGTGCAT-3′ (SEQ ID NO: 1) and 5′-gtgtcgaCTAAAGAAGACGTCCTCTGCATTCAAT-3′ (SEQ ID NO: 2). The PCR product was ligated into SacI-SalI sites in pEGFP-C3. Site directed mutagenesis to generate SETDB1 H1224K was performed by PCR using the above two oligonucleotides and the oligonucleotides: 5′-GGGCCGCTACCTCAACaagAGTTGCAGCCCCAACC-3′ (SEQ ID NO: 3) and 5′-GGTTGGGGCTGCAACTcttGTTGAGGTAGCGGCCC-3′ (SEQ ID NO: 4). Cloning procedures were confirmed by DNA sequencing. pcDNA-HDAC6-FLAG was Addgene plasmid #30482. EB1-GFP, pEGFP-Moesin were also used.

Protein lysate preparation and Western blot analysis: For nuclear-cytoplasmic fractionation, cells were washed in PBS, re-suspended in STM buffer: 50 mM Tris pH 7.4, 250 mM sucrose, 5 mM MgSO₄, 10 mM NaF, 2 mM DTT, 0.025% Triton X-100 and 1× protease inhibitor cocktail (539134, Millipore, Burlington, MA, USA) and lysed by a Dounce homogenizer. Following cell membrane breakage (as monitored under the microscope), the nuclei fraction was precipitated at 700 g for 10 minutes at 4° C. The cytoplasmic supernatant was collected to a new tube and the nuclear pellet was re-suspend in TP buffer: 10 mM Tris pH 7.4, 10 mM phosphate buffer pH 7.4, 5 mM MgSO₄, 10 mM NaF and 1× protease inhibitor cocktail supplemented with 5% glycerol. Protein concentrations were measured using the Bradford assay. Samples were stored at −80° C.

For whole cell extract cells were washed in PBS, re-suspended in 2×SDS sample buffer (100 mM Tris pH 6.8, 10% glycerol, 2% SDS, 100 mM bromophenol blue, 0.1 M DTT and 1× protease inhibitor cocktail) and sonicated. Samples were then heated at 95° C. for 10 minutes and kept at −20° C. until usage.

Protein extracts were separated in SDS-PAGE and analyzed by Western blot analysis using the following antibodies: rabbit anti-SETDB1 (1:500, Santa Cruz Biotechnology sc-66884), rabbit anti-SUV39H2 (1:1,000, Abcam ab190270), rabbit anti-histone H3 (1:10,000, Millipore 05-928), mouse anti-α-tubulin (1:5,000, Thermo Fisher Scientific 62204), mouse anti-acetylated tubulin (1:1,000, Santa Cruz Biotechnology sc-23950), mouse anti-β-actin (1:5,000, Sigma-Aldrich A1978), mouse anti-GFP (1:500, Santa Cruz Biotechnology sc-9996) and rabbit anti-FLAG (1:1,000, Sigma-Aldrich F7425). Quantitative data analysis was performed with ImageJ/Fiji software (National Institute of Health, Bethesda, USA).

Co-immunoprecipitation: Cells were harvested 48 hours following co-transfection and lysed in extraction buffer (50 mM Tris pH 8, 150 mM NaCl, 20 mM EDTA, 50 mM NaF, 1% Triton X-100) supplemented with 1× protease inhibitor cocktail. Cells were lysed on ice for 30 minutes and centrifuged at 20,000 g for 30 minutes at 4° C. A 5% input control sample was taken from each cleared lysate and boiled in SDS sample buffer for 10 minutes at 98° C. For immunoprecipitation, clarified lysates were supplemented with GFP Trap Magnetic Agarose (gtma-20, Chromotek, Planegg-Martinsried, Germany) and incubated with rotation for 1 hour at 4° C. The beads were washed once in extraction buffer and three times in PBS and boiled in SDS sample buffer for 10 minutes at 98° C., before being loaded on an 8% acrylamide gel for subsequent Western blot analysis.

Immunostaining: Cells plated on fibronectin-coated coverslips (03-090-1-05, Biological Industries, Beit-Haemek, Israel) were fixed in methanol supplemented with 1 mM EGTA at −20° C. for 6 minutes. Antibodies included rabbit anti-SETDB1 (1:50, Santa Cruz Biotechnology sc-66884), rabbit anti-SETDB1(1:500, Cell signaling Technology 93212), rabbit anti-SUV39H2 (1:120, Abcam ab190270), mouse anti-α-tubulin (1:200, Thermo Fisher Scientific 62204), goat anti-GFP (1:2,000, Abcam ab5450) and rabbit anti-7-tubulin (1:400, Abcam ab1132). All images were collected using an Olympus 1X81 fluorescent microscope with a coolSNAP HQ2 CCD camera (Photometrics, Tucson AZ, USA).

MT recovery assay: Cells plated on fibronectin-coated coverslips were treated with 7 μg·ml⁻¹ of nocodazole for 3 hours. Following three washings with cold DMEM to remove the nocodazole, the cells were incubated at 37° C. in pre-warmed complete growth medium for the indicated periods of time, fixed and immunostained as described above. Quantitative data analysis was performed with ImageJ/Fiji software (NHI) by manual delineation of the total area covered by MTOC-linked MTs.

Time-lapse imaging: For live imaging cells were plated in 35-mm glass-bottom dishes. Time-lapse images were collected with a coolSNAP HQ2 CCD camera (Photometrics, Tucson AZ, USA) mounted on an Olympus 1X81 fluorescent microscope at 37° C. and 7% CO₂. Frames were captured every 3 seconds for 5 minutes-movies to track growing MT ends and every 3 minutes for 10 hours-movies to monitor mitotic progression. Acquired images were analyzed by ImageJ/Fiji software. To analyze growing MT ends, MTrackJ plugin was used to track EB1-GFP comets. Comets were analyzed in each frame considering the distal site of the comet as the comet point. Data analysis was done according to duration and length tracked. To analyze mitosis progression, mitotic events were followed in terms of time and success rate.

### Example 1: SETDB1 Associates with the MT Network

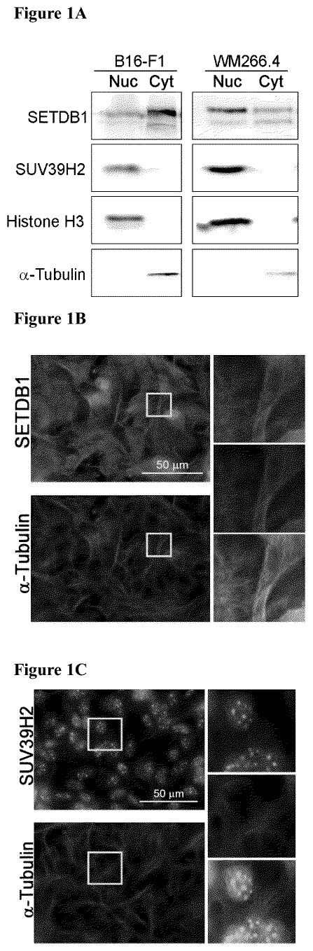

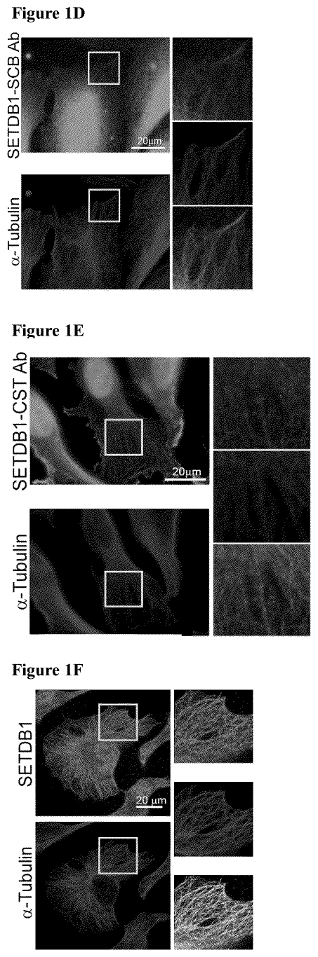

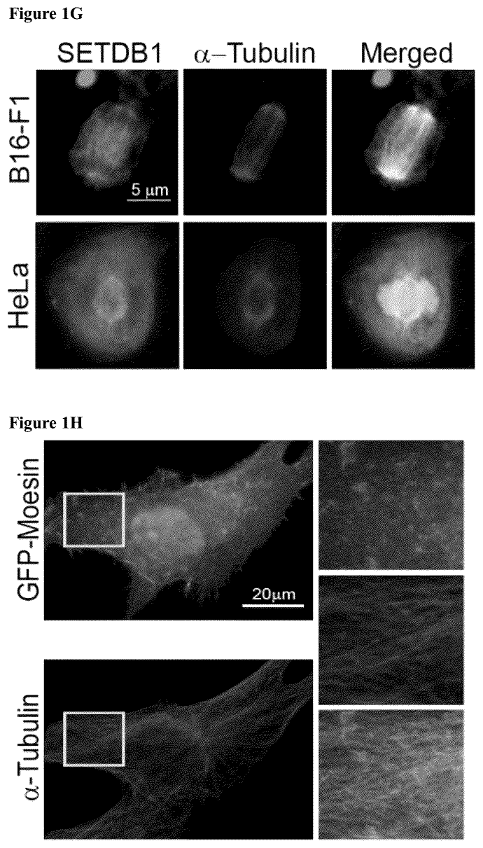

To identify a cytoplasmic role for SETDB1, its cytoplasmic localization was first verified in both mouse and human melanoma cells: B16-F1 and WM266.4 cells, respectively. Biochemical fractionation followed by Western blot analysis identified a substantial amount of SETDB1 in the cytoplasmic fraction of these cells in contrast to another H3K9 methyltransferase, SUV39H2 which was found only in the nuclear fraction (FIG. 1A). Immunostaining for SETDB1 verified this observation and reveled partial co-localization of SETDB1 with MTs (FIG. 1B-C). To validate the immunostaining a second antibody against SETDB1 was used which revealed a similar pattern of localization in human WM266.4 cells (FIG. 1D-E). Co-localization of SETDB1 with microtubules was found also in HeLa cells (FIG. 1F). Moreover, this pattern was maintained during mitosis as well (FIG. 1G). To verify these results, cells over-expressing GFP-fused SETDB1 were analyzed and a similar pattern of partial co-localization with MTs was revealed (FIG. 1H-I). Finally, control and GFP-SETDB1 over-expressing HEK293 cells were used for immunoprecipitation with anti GFP antibodies. GFP-SETDB1 immunoprecipitated alpha tubulin indicating a stable interaction between SETDB1 and tubulin (FIG. 1J). These results demonstrate SETDB1 association with MTs in several cell types both in interphase and mitosis, suggesting a role for SETDB1 in MT function.

### Example 2: SETDB1 Affects MT Growth Rate

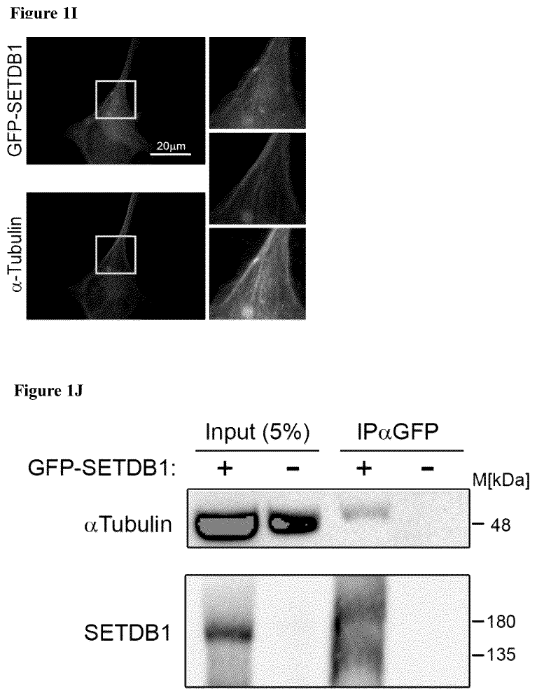

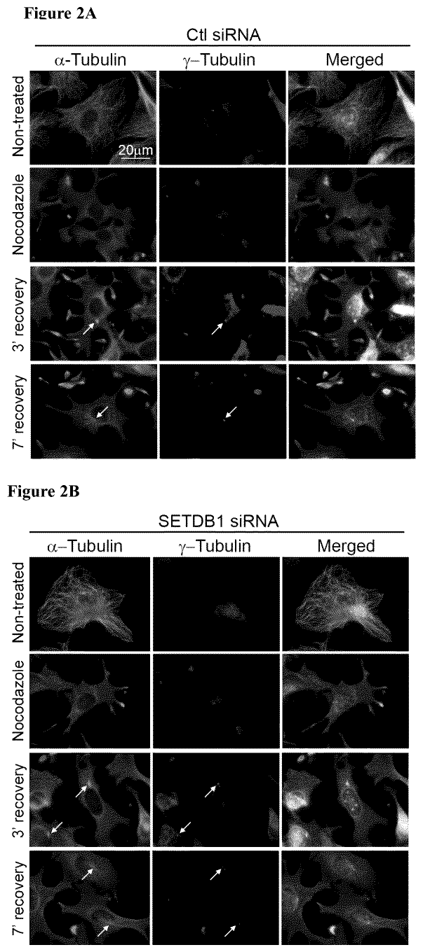

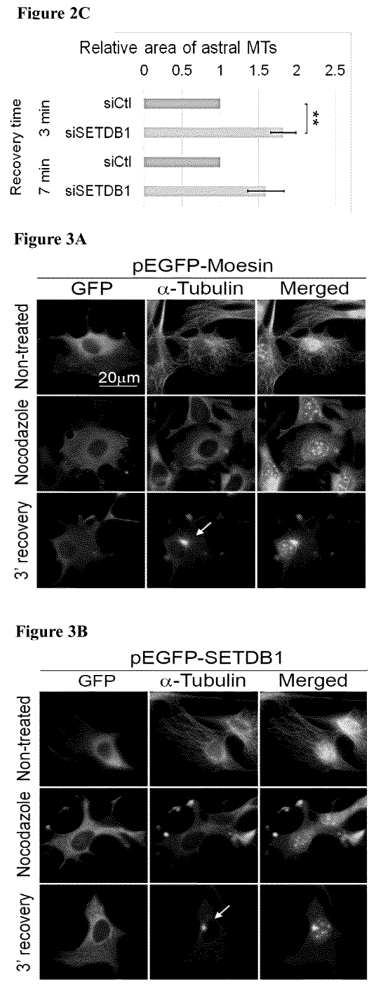

To evaluate whether SETDB1 can affect MT functions, the impact of SETDB1 silencing on the rate of MT growth during recovery from nocodazole treatment was tested. B16-F1 cells were transfected with control siRNA or SETDB1 siRNA and treated with nocodazole for 3 hours to depolymerize MTs. Following nocodazole washout the cells were further incubated for additional 3 or 7 minutes, fixed and immunostained for α-tubulin. Measurements of the area covered by MTs originated from the MTOC revealed an increase of 82% and 60% in SETDB1 KD cells in comparison to control cells at the 3 minutes and 7 minutes time points, respectively (FIG. 2A-C). Thus, suggesting SETDB1 attenuates MT growth rate.

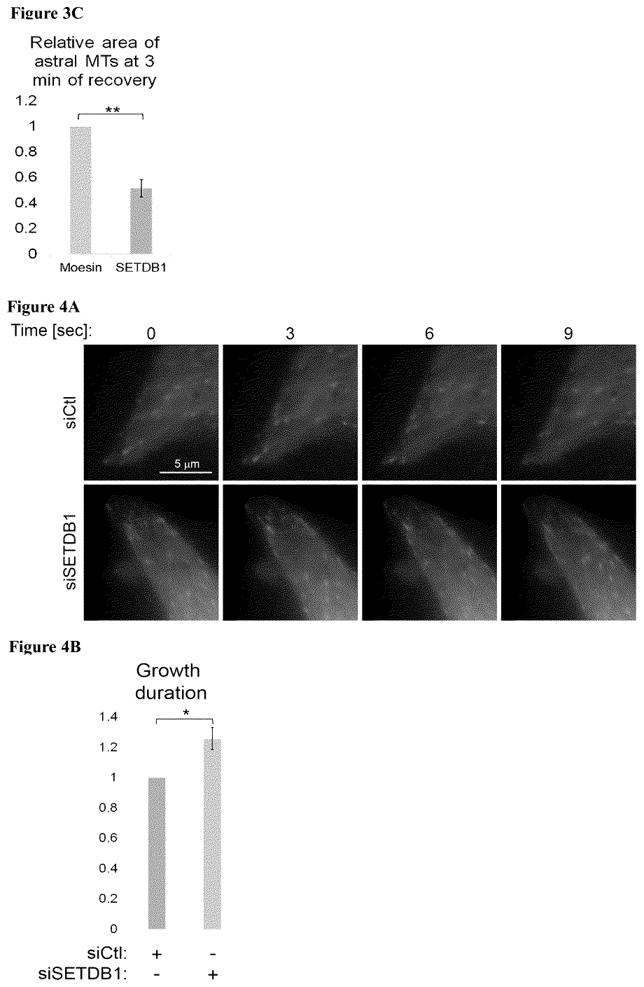

SETDB1 KD reduces the protein levels in both the cytoplasm and the nucleus, thus the KD effects may be due to reduced nuclear activity of SETDB1 as a transcriptional regulator or due to reduced association of cytoplasmic SETDB1 with the MT network. Notably, over-expressed (OE) SETDB1 has been reported to localize to the cytoplasm and this was indeed observed (FIG. 1H, 3B). Therefore, the MT recovery assay was repeated after nocodazole treatment with cells over-expressing GFP-SETDB1 or GFP-Moesin that served as a negative control. As shown in FIG. 3A-C, the levels of MTOC-linked MTs 3 minutes after nocodazole washout in GFP-SETDB1 OE cells were reduced in half compared to GFP-Moesin OE cells. This effect was in the opposite direction of the SETDB1 silencing effect (FIG. 2A-C), suggesting SETDB1 effect on MT growth is due to the activity of its cytoplasmic pool rather than the nuclear SETDB1.

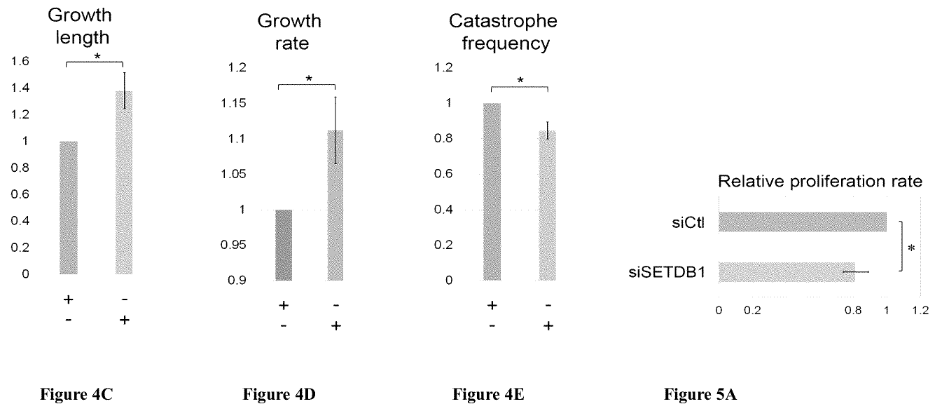

To verify the effects of SETDB1 on MT growth, the MT plus end dynamics were monitored by tracking GFP-fused EB1 in SETDB1 KD cells (FIG. 4A-E). Notably, MT growth duration, growth length and growth rate were increased in SETDB1 KD cells by 25%, 38% and 11%, respectively, in comparison to control cells (FIG. 4B-D). On the other hand, MT catastrophe rate in SETDB1 KD cells was reduced by 15% in comparison to control cells (FIG. 4E). These results suggest that SETDB1 is a negative regulator of MT growth.

### Example 3: SETDB1 is Important for Proper Cell Division

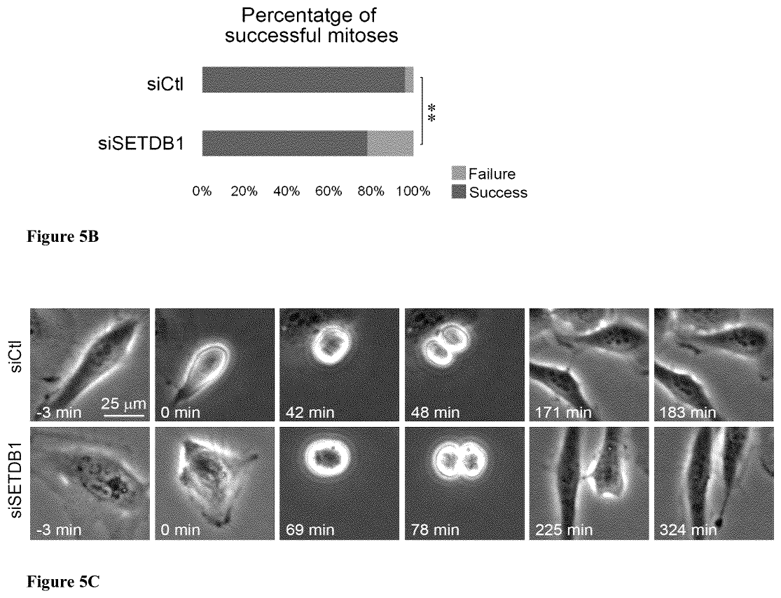

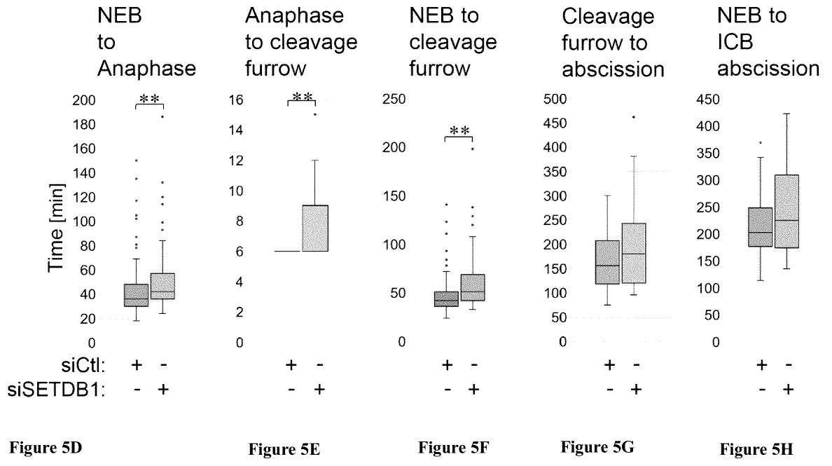

The finding that SETDB1 regulates MT dynamics led to testing if SETDB1 is important for cell division, a process that is heavily dependent on MT organization. Indeed, SETDB1 KD attenuated the cellular proliferation rate by 20% (FIG. 5A) while increasing unsuccessful mitotic events from 4.1% to 22.1% (FIG. 5B). Measuring the lengths of the different cell division stages in cells that were able to finish mitosis successfully revealed a significant increase in the duration of both early and late mitosis in SETDB1 KD cells in comparison to control cells: in SETDB1 KD cells the durations from nuclear envelope breakdown (NEB) to anaphase and from anaphase to the appearance of a cleavage furrow were increased by 17% and 40%, respectively, in comparison to control cells (FIG. 5C-H).

### Example 4: SETDB1 Affects MT Growth Rate Independently of its Methyltransferase Activity

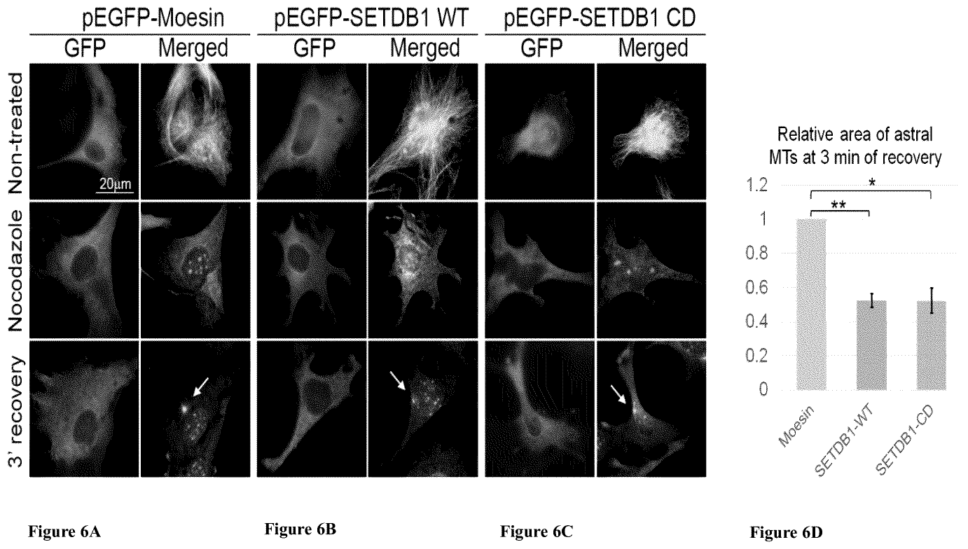

SETDB1 is a well-established methyltransferase that was found to methylate both histones and non-histone proteins. Moreover, recently α-tubulin was found to be methylated at lysine 40 by SETD2. To evaluate if the effect of SETDB1 on MT growth rate is dependent on its methyltransferase activity the MT recovery assay was repeated while over-expressing either WT SETDB1 or H1224K point mutated SETDB1 (FIG. 6A-D). The H1224K mutation was shown before to impair completely the methyltransferase activity of SETDB1. As shown in FIG. 6A-D, over-expression of the enzymatically inactive H1224K SETDB1 attenuated MT recovery from nocodazole treatment to the same extent as over-expression of WT SETDB1. Thus, SETDB1 can affect MT dynamics by a mechanism that is independent of its enzymatic activity.

### Example 5: SETDB1 Affects Tubulin Acetylation

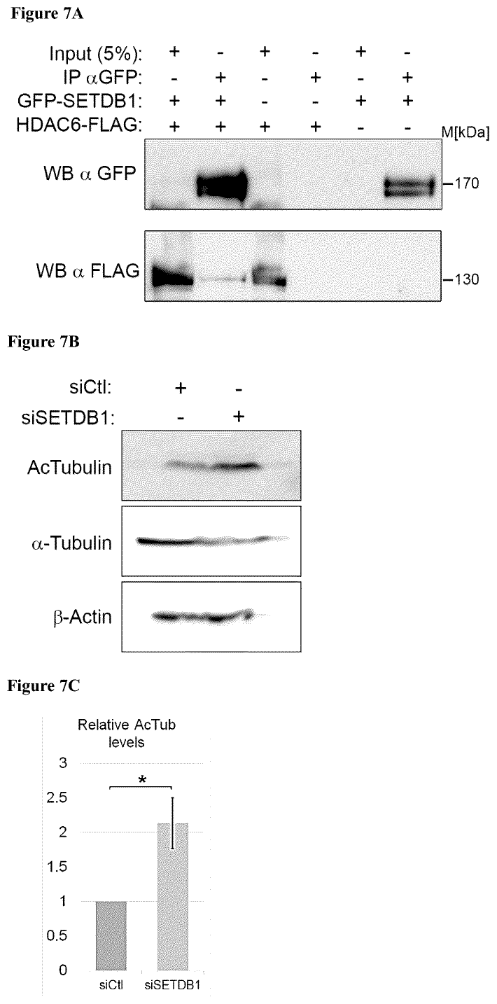

The molecular mechanism by which SETDB1 affects MT dynamics seemed to be independent of its methyltransferase activity. To identify the molecular mechanism by which SETDB1 affects MT dynamics two features were taken into account: the first is that nuclear SETDB1 is known to work in a complex with HDAC1 and HDAC2 to repress transcription; the second is that acetylation of Lys40 in α-tubulin (acetylated tubulin) is a key tubulin post-translational modification, which is associated with stable MTs in various cellular contexts. It was therefore hypothesized that SETDB1 may interact with a tubulin HDAC and affect its activity. Since the major tubulin deacetylase is HDAC6, it was tested if SETDB1 can interact with it and affect tubulin acetylation levels. As shown in FIG. 7A, HDAC6 co-immunoprecipitated with SETDB1, suggesting an in vivo interaction between these two factors. In addition, SETDB1 KD led to an increase of 110% in the levels of tubulin acetylation (FIG. 7B-C). This supports the hypothesis that SETDB1 serves as a co-factor of HDAC6 in the cytoplasm to support tubulin deacetylation to regulate MT dynamics.

### Example 6: Assessing the Effect of SETDB1 on Cell Sensitivity to Microtubule-Targeting Agents

Microtubule-targeting agents (MTAs), like taxanes and vinca alkaloids are commonly used in cancer therapy, however, tumor resistance to MTAs is a major problem. To overcome this problem new MTAs are developed and combination therapies of MTAs with other drugs such as immunotherapies are considered. Since SETDB1 is both over-expressed in various cancer types and affects MT dynamics its involvement in MTAs resistance is tested by evaluating the effect of altered SETDB1 levels on the potency of the MTAs paclitaxel (an MT stabilizing drug) and vinblastine (an MT disrupting drug). HeLa and MDA-231 cells with altered SETDB1 levels (both overexpressing and knocked down/out) are used. Cells are tested in regular monolayer growth and in 3D spheroids embedded in collagen gels. Spheroids are generated by plating the cells on top of agarose in a 96 well plate for 48 or 72 hours for HeLa cells or MDA-231 cells, respectively. To better mimic in vivo conditions the spheroids are embedded in collagen gels. Drugs are added 1 hour later, once the collagen is polymerized. The various cells are treated for 72 hours with various concentrations of paclitaxel and vinblastine to calculate IC50. Cell viability is measured by counting trypsinized cells mixed with Trypan blue. Spheroids are treated with collagenase first to produce a single cell suspension. In addition, apoptosis levels and cell cycle progression are monitored in the various cells after 48 hours of treatment with the IC50 of each drug as determined in control cells. Apoptosis levels are determined by FACS analysis of fluorescent annexin V binding and cell cycle progression is determined by FACS analysis of propidium iodide-stained cells.

The SETDB1 effects on tumor cell response to MTAs are confirmed in an animal model. Tumors are generated in nude mice by administering breast cancer MDA-231 cells with altered SETDB1 levels. Paclitaxel and vinblastine are administered, and drug effectiveness is monitored.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of determining suitability of a subject in need thereof to be treated with a microtubule targeting agent (MTA), the method comprising receiving a sample from said subject, measuring SETDB1 expression in said sample and determining suitability based on said SETDB1 expression, thereby determining suitability of a subject to be treated with an MTA, the method further comprises administering said MTA to a suitable subject.

2. The method of claim 1, wherein said MTA is a microtubule (MT) stabilizing agent and wherein any one of: (i) expression of SETDB1 above a predetermined threshold indicates said subject is unsuitable for treatment with said MTA; and (ii) expression of SETDB1 at or below a predetermined threshold indicates said subject is suitable for treatment with said MTA, optionally wherein said predetermined threshold is an expression level in a healthy control.

3. (canceled)

4. The method of claim 2, wherein said MT stabilizing agent is selected from plant-based MT stabilizing agent, a bacterial MT stabilizing agent and a small molecule stabilizing agent.

5. The method of claim 2, wherein said MT stabilizing agent is selected from a taxane, a taccalonalide, an epothilone, ceratamine A, ceratamine B, FR182877, dictyostatin, discodermolide, KS-1-199-32, eleutherobin, sarcodictyin, A, sarcodictyin B, zampanolide, dactylolide, laulimalide, isolaulimalide, peloruside A, persin, GS-164, Synstab, 4′-methoxy-2-styrylchromone, (Z)-1-(2-bromo-3, 4, 5-trimethoxyphenyl)-3-(3-hydroxy-4-methoxyphenylamino)prop-2-en-1-one, an a-cyano bis(indolyl)chalcone, a dienone and a derivative thereof, optionally wherein any one of (i) said taxane is selected from paclitaxel, paclitaxel C, docetaxel, abraxane, cabazitaxel, larotaxel tesetaxel, TPI-287, 10-deacetylbaccatin III, baccatin III, and 7-epipaclitaxe and derivatives thereof; (ii) said taccalonalide is selected from taccalonalide A, B, E, H2, I, J, N, R, T, Z, AA, AB, AC, AD, AF, AJ, AK, AL, AM, AN and AO: (iii) said epothilone is selected from epothilone A, epothilone B, epothilone C, epothilone D, epothilone E, ixabepilone, sagopilone, patupilone, BMS-310705, KOS-1584 and BMS-753493.

6. (canceled)

7. (canceled)

8. (canceled)

9. The method of claim 2, wherein said MT stabilizing agent is an HDAC6 inhibitor.

10. The method of claim 1, wherein said MTA is an MT destabilizing agent and wherein any one of: (i) expression of SETDB1 above a predetermined threshold indicates said subject is suitable for treatment with said MTA; and (ii) expression of SETDB1 at or below a predetermined threshold indicates said subject is unsuitable for treatment with said MTA, optionally wherein said predetermined threshold is an expression level in a healthy control.

11. (canceled)

12. The method of claim 10, wherein said MT destabilizing agent is selected from plant-based MT destabilizing agent, a bacterial MT destabilizing agent and a small molecule destabilizing agent.

13. The method of claim 10, wherein said MT destabilizing agent is selected from a vinca alkaloid, maytansine, disorazole Z, a lactone, circumin, colchicine, combretastatin, TH588, chalcone, podophyllotoxin, indibulin, is 2-methoxyestradiol, pironetin, noscapinoid, noscapine, 9-bromonoscapine, quercetin and derivatives thereof, optionally wherein any one of (i) said vinca alkaloid is selected from vinblastine, vincristine, colcemid, nocodazole, vindesine, vinorelbine, vincaminol, vineridine, vinburnine, vinpocetine, vinflunine, minovincine, methoxyminovincine, minovincinine, vincdifformine, desoxyvincaminol, crypthophycin1, romidepsin (FK-228), halichondrin B, erilubin, soblidotin, dolastin 15, dolastin 10, and vincamajine; (ii) said lactone is selected from plocabulin (PM060184), emtansine, rhizoxin, and spongistatin; and (iii) said derivative of combretastatin is selected from combretastatin A-1, combretastatin A-4, fosbretabulin, Oxi4503, combretastatin 4-O-phosphate (CA-4P), ombrabulin.

14. (canceled)

15. (canceled)

16. (canceled)

17. The method of any claim 10, wherein said MT destabilizing agent is HDAC6 or a functional fragment thereof.

18. (canceled)

19. The method of claim 1, wherein said subject suffers from a disease, disorder or condition treatable by an MTA, optionally wherein said disease is a proliferative disease, further optionally wherein said proliferative disease is cancer.

20. (canceled)

21. (canceled)

22. (canceled)

23. The method of claim 19, wherein said cancer is selected from brain, skin, breast, lung, renal, liver, pancreatic, head and neck, hematopoietic, endometrial, bladder, sarcoma, glioma, colorectal, gastric, prostate, ovarian, testicular, and cervical cancer.

24. The method of claim 1, wherein said sample is a tumor sample, comprises tumor cells or comprises cell free DNA from a tumor cell.

25. The method of claim 1, wherein said expression is mRNA expression or protein expression.

26. The method of claim 1, wherein said SETDB1 expression is cytoplasmic SETDB1 expression.

27. (canceled)

28. (canceled)

29. The method of claim 1, wherein said MTA is a microtubule destabilizing agent and further comprising enhancing HDAC6 expression, function or both in said suitable subject.

30. The method of claim 1, wherein said MTA is a microtubule stabilizing agent and further comprising decreasing HDAC6 expression, function or both in said suitable subject.

31. The method of claim 29, wherein said HDAC6 expression or function is within a cancer cell.

32. A method of treating a subject suffering from a SETDB1-associated cancer, the method comprising ii) reducing microtubule stability in said cancer characterized by increased expression and (ii) increasing microtubule stability in a cancer characterized by decreased or healthy SETDB1 expression, thereby treating said subject.

33. The method of claim 32, wherein any one of: (i) said reducing comprises administering an MTA that destabilizes microtubules, optionally wherein said reducing comprises increasing HDAC6 expression, function or both in said cancer; and (ii) said increasing comprises administering an MTA that stabilizes microtubules, optionally wherein said increasing comprises decreasing HDAC6, expression, function or both in said cancer.

34. (canceled)

35. (canceled)

36. (canceled)

37. (canceled)

38. (canceled)

39. A kit comprising:
a. a microtubule targeting agent (MTA); and
b. an agent for specific detection of SETDB1 expression.

40. (canceled)

41. (canceled)

42. (canceled)


SEQUENCE LISTING (6 sequences)

SEQ ID NO: 1 - DNA, 31 bp, synthetic construct
  misc_feature 1..31: note = Synthetic
  source 1..31: mol_type = other DNA
cagagctcatgtcttcccttcctgggtgcat

SEQ ID NO: 2 - DNA, 34 bp, synthetic construct
  misc_feature 1..34: note = Synthetic
  source 1..34: mol_type = other DNA
gtgtcgactaaagaagacgtcctctgcattcaat

SEQ ID NO: 3 - DNA, 35 bp, synthetic construct
  misc_feature 1..35: note = Synthetic
  source 1..35: mol_type = other DNA
gggccgctacctcaacaagagttgcagccccaacc

SEQ ID NO: 4 - DNA, 35 bp, synthetic construct
  misc_feature 1..35: note = Synthetic
  source 1..35: mol_type = other DNA
ggttggggctgcaactcttgttgaggtagcggccc

SEQ ID NO: 5 - DNA, 23 bp, synthetic construct
  misc_feature 1..23: note = Synthetic
  source 1..23: mol_type = other DNA
atgtcttcccttcctgggtgcat

SEQ ID NO: 6 - DNA, 27 bp, synthetic construct
  misc_feature 1..27: note = Synthetic
  source 1..27: mol_type = other DNA
ctaaagaagacgtcctctgcattcaat